(19) United States
(12) Patent Application Publication
(10) Pub. No.: US 2015/0065893 A1
(43) Pub. Date: Mar. 5, 2015

(54) WEARABLE ELECTRONIC DEVICE, CUSTOMIZED DISPLAY DEVICE AND SYSTEM OF SAME

(71) Applicant: CYWEE GROUP LIMITED, TORTOLA (VG)

(72) Inventor: Zhou Ye, Foster City, CA (US)

(73) Assignee: CYWEE GROUP LIMITED, TORTOLA (VG)

(21) Appl. No.: 14/011,734

(22) Filed: Aug. 27, 2013

(51) Int. Cl. A61B 5/00; H04M 1/725; A61B 5/11; A61B 5/0205; A61B 5/021

(52) U.S. Cl. CPC A61B 5/6898; A61B 5/0205; A61B 5/021; A61B 5/11; A61B 5/681; A61B 5/742; A61B 5/7475; A61B 5/0022; H04M 1/7253; H04M 1/72583; A61B 5/02438
USPC 600/483; 600/300; 600/595; 455/566; 455/41.2

(57) ABSTRACT

Cellular telephone set having a wearable electronic device and a display device is disclosed. Wearable electronic device includes a first processor, a first storage memory, a small display, a small battery pack, a cellular communication module, a first local communication module, a character receiving unit, a first charging circuitry, a motion sensor, and a biosensor configured for generating physiological signals indicating health conditions of user. Display device includes second processor, second storage memory, large display, large battery pack, a second local communication module, a character input unit, video processing unit; and second charging circuitry. Wearable electronic device is worn on a wrist of user and capable to perform cellular phone functions by itself without display device. Display device is configured to provide a full QWERTY keyboard to facilitate data entry into wearable electronic device. Display device is capable of charging wearable electronic device when connected in wired or wireless manner.

### FIELD OF THE INVENTION

The present disclosure relates to a wearable electronic device, and particularly to a cellular telephone set including a wearable electronic device and a display device working together with the wearable electronic device.

### BACKGROUND OF THE INVENTION

Nowadays smartphones are typically configured and equipped with communication, entertainment, data input, display, and sensor functionalities. The communication functionalities of the smartphones are for voice/data services in accordance with cellular communication technical standards such as GSM/EDGE/CDMA/WCDMA/HSPA/LTE network technologies, for establishing cellular connection to base stations provided by operators (e.g. Verizon, AT&T, China Mobile, . . . etc.), and using WIFI or Bluetooth for providing local wireless connection with peripherals (e.g. Wifi hotspot, Bluetooth headset . . . etc.).

Entertainment functionalities for the smartphone includes for example, mobile gaming, multimedia playback (audio reproduction, video reproduction, radio service . . . etc.), and internet browsing. Data and/or character-input are provided in the form of a physical or virtual QWERTY keyboard (see FIG. 1), or a phone pad with numbers and letters (see FIG. 2). Display functionality is realized by a display panel of the size of 2.8 inches to 6 inches with optional touch panel capability, which may provide a virtual QWERTY keyboard to facilitate data and/or character input. Sensors which are configured inside conventional smartphones include for example, ambient light sensor (ALS), proximity sensor, accelerometer, gyroscope, magnetometer, etc. Ambient light sensor (ALS) is used to detect the amount of light available in the ambient environment, and can be used to help the smartphone to adjust its display brightness so as to extend battery life and enable easy-to-view displays that are optimized to the environment. Proximity sensor detects how close the screen of the smartphone is in relation to the user's ear, cheek, or face. When the smartphone is brought at close proximity to the user's ear/cheek/face, the smartphone can automatically turn off its display to save battery life, and/or disable touch sensitive panel to prevent unintentional touches, and/or pause multimedia playback activity (e.g. playing music or video). When the phone is removed from the user's ear/cheek/face, the phone may resume its previous activity. Accelerometer is a motion sensor that measures acceleration, which allows the phone to detect the viewing orientation of the display on the smartphone and adapts the displayed content to suit changes in viewing orientation, and can also be used to control third-party apps, including for example games. A gyroscope is a motion sensor for measuring or maintaining orientation, based on the principles of angular momentum, and is used for finding the position and orientation of the smartphone. Magnetometer is a sensor to measure the strength and, in some cases, the direction of magnetic field, which allows the smartphone to be used as a compass or to know the direction in various applications, such as navigation.

While smartphones are very popular in the marketplace, wearable electronic devices have also emerged. Pebble, for example, is an electronic watch configured for smartphones with a black and white rectangular e-paper display. Pebble is equipped with a vibrating motor, magnetometer, ALS sensor, accelerometer with gesture detection, battery, support for Bluetooth, and is water-resistant, and has some functional specific apps (i.e., software for mobile application). Pebble uses an OS (Operation System) based on FreeRTOS. Uses for Pebble include receiving notifications for incoming calls, text, and email messages and alerts from interne, acting as a remote control for providing controls to smartphone, displaying sport activity information updates, etc. As a smart watch, Pebble is able to provide two-way communication with the smartphone.

The existing technologies have a few disadvantages. To facilitate data and/or character input and enhance multimedia experiences for the user, a large display with the capability of providing virtual QWERTY keyboard is desired. With a large display, the smartphone becomes larger and is thus less portable. Wrist-mounted portable cellular telephone has been proposed, but its small display cannot provide satisfactory data entry and multimedia experiences. In addition, while touch or multi-touch gesture operation becomes popular in smartphones with touch panel display, the small display of the wrist-mounted portable telephone is inappropriate to provide convenient touch or multi-touch gesture operations. Further improvements of the existing technologies are desired.

### SUMMARY OF THE INVENTION

It is one objective of the present invention to provide a cellular telephone set including a wearable electronic device and a display device. The wearable electronic device is physically configured to be worn by a user, for example, on his/her wrist, and is capable for the user to take incoming calls and make outgoing calls by itself without using the display device. The display device has a larger display comparing to the display of the wearable electronic device, and may provide better data entry and multimedia enjoyment experiences to the user.

In one embodiment, the wearable electronic device includes a first display, a cellular communication module, and a first local communication module. The display device includes a second display with touch panel capability, and a second local communication module for providing local communication with the first local communication module of the wearable electronic device. The display device is configured to provide a virtual QWERTY keyboard on the second display, so as to facilitate data entry by the user.

The wearable electronic device may further include a biosensor for detecting the user's body and generating physiological signals, which may be accumulated and analyzed to generate health information of the user. The health information may include, for example, the user's heart rate or blood pressure. The health information may be shown on the wearable electronic device in a first format, which is simpler and may contain less information so as to be properly shown on the first display of the wearable electronic device. The health information may also be shown on the second display of the display device in a second format, which is more content-rich and contains more detailed information to the user. For example, on the wearable electronic device, only the latest detected health information of the user is shown, and on the display device, historical health information is shown to the user as well. The second display of the display device may also show graphical illustration of the health information to the user.

The biosensor may utilize Nanosecond Pulse Near-field Sensing (NPNS) technology to detect the user's body and generate the physiological signals.

To obtain more reliable and accurate health information, the wearable electronic device may further include a motion sensor to detect movement of the wearable electronic device. If the wearable electronic device is in an environment suitable for physiological signal detection (e.g. when the wearable electronic device is stationary), the biosensor may be enabled to detect the body and generate the physiological signals.

The wearable electronic device may be configured to display time, text messages, icons indicating status of the wearable electronic device, or icons indicating status of the display device.

The display device may include a large battery pack, so it may provide charging to the wearable electronic device in a wired or wireless manner. The wired manner may be through, for example, a micro USB charging interface. The wireless manner may be through, for example, inductive charging.

It is also an objective of the present invention to provide a cellular telephone set including a wearable electronic device and a display device. The wearable electronic device is physically configured for wearing by the user, and is capable for the user to take incoming calls and make outgoing calls by itself without using the display device. The wearable electronic device further includes a biosensor for detecting the user's body and generating physiological signals. The physiological signals may be accumulated and analyzed to generate the user's health information. The health information may include, for example, the user's heart rate or the blood pressure. The accumulation and/or analysis of the physiological signals may be performed on the wearable electronic device or on the display device to generate the user's health information. The user's health information may be transmitted between the wearable electronic device and the display device, and be simultaneously shown on the wearable electronic device and the display device in a different format.

In one embodiment, the biosensor may utilize Nanosecond Pulse Near-field Sensing (NPNS) technology to detect the user's body and generate the physiological signals.

The wearable electronic device and the display device may be connected by point-to-point wireless communication, for example, Wifi Direct or Bluetooth standard. The health information may be displayed on the wearable electronic device in a first format, and displayed on the display device in a second format different from the first format. The second format may contain more information, for example, the historical record of the same user's health information, which is obtained previously over a certain period of time.

The display device may further include a display with touch panel capacity. The display device may provide a virtual QWERTY keyboard on the display to facilitate data entry by the user to the wearable electronic device. The display device may provide touch and/or multi-touch gesture operations for the user to control the display device itself, or control the wearable electronic device through the point-to-point wireless communication.

It is another objective of the present invention to provide a cellular telephone set including a wearable electronic device and a display device. The wearable electronic device is physically configured for wearing by the user, and is capable for the user to take incoming calls and make outgoing calls by itself without using the display device. The display device forms an independent body from the wearable electronic device, and has a greater video processing capability than the wearable electronic device.

According to one embodiment, the display device includes a touch panel display, and may provide a virtual QWERTY keyboard for data and/or character entry by the user to the wearable electronic device. The display device may provide touch and/or multi-touch gesture operations for the user to control the display device itself (for example, control the display device to zoom-in or zoom-out a photo when the display device is showing a series of photos), or control the wearable electronic device through the point-to-point wireless communication (for example, control the wearable electronic device to pause or fast forward when the wearable electronic device is reproducing an audio information).

The wearable electronic device may further include a motion sensor and a biosensor. The motion sensor is configured to detect movement of the wearable electronic device. If the wearable electronic device is in an environment suitable for physiological signal detection (e.g. when the wearable electronic device is stationary), the biosensor is enabled to detect the user's body and generate physiological signals. The physiological signals could be accumulated and analyzed on the wearable electronic device to obtain health information. The health information could be displayed on the wearable electronic device, or transmitted to the display device for display on the display device with more details. The physiological signals could also be accumulated and analyzed on the display device to obtain the health information, and the health information could be displayed on the display device and also transmitted to the wearable electronic device for display on the wearable electronic device with less details.

### BRIEF DESCRIPTION OF THE DRAWINGS

The present invention is illustrated by way of example and not limitation in the figures of the accompanying drawings in which like references indicate similar elements. Besides, many aspects of the disclosure can be better understood with reference to the following drawings. Moreover, in the drawings like reference numerals designate corresponding elements throughout. Wherever possible, the same reference numerals are used throughout the drawings to refer to the same or like elements of an embodiment.

FIG. 1 shows a physical or virtual QWERTY keyboard for data entry for embodiments of present invention.

FIG. 2 shows a phone pad with numbers and letters for data entry for embodiments of present invention.

FIG. 3 shows an integrated electronic system of a first embodiment which includes a wearable electronic device and a customized display device.

FIGS. 4A and 4B show block diagrams of the wearable electronic device and the customized display device of a first embodiment, in which many of the components have been omitted in FIG. 4B to highlight the power charging connections and configurations of the wearable electronic device and the customized display device.

FIGS. 5A and 5B show block diagrams of the wearable electronic device and the customized display device of a second embodiment, in which many of the components have been omitted in FIG. 5B to highlight the power charging connections and configurations of the wearable electronic device and the customized display device.

FIG. 6 shows a flowchart illustrating an embodiment of a method to obtain the user's health information using the wearable electronic device.

FIG. 7 shows a flowchart illustrating another embodiment of a method to obtain the user's health information using the wearable electronic device.

FIGS. 8A and 8B show user's health information being graphically shown on the large display of the customized display device, with FIG. 8A showing a detailed blood pressure versus date and time chart, and FIG. 8B showing blood pressure and pulse histogram bar charts.

FIG. 9 shows user's health Information shown in a more simplified and representative manner on the wearable electronic device.

FIG. 10 shows a flowchart illustrating an embodiment for a method for performing character input for controlling various operating functions of the wearable electronic device.

### DETAILED DESCRIPTION OF PREFERRED EMBODIMENTS

The present invention will now be described more specifically with reference to the following embodiments. It is to be noted that the following descriptions of preferred embodiments of this invention are presented herein for purpose of illustration and description only. It is not intended to be exhaustive or to be limited to the precise form disclosed.

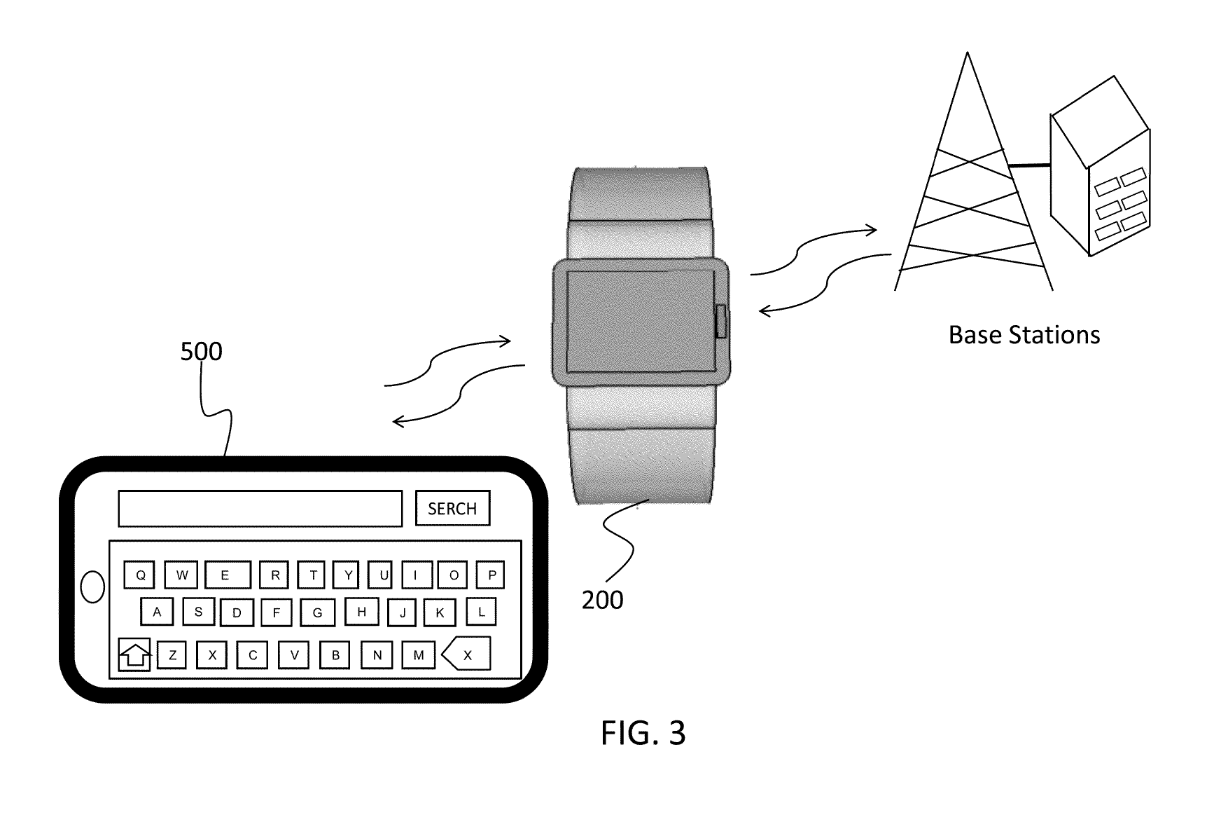

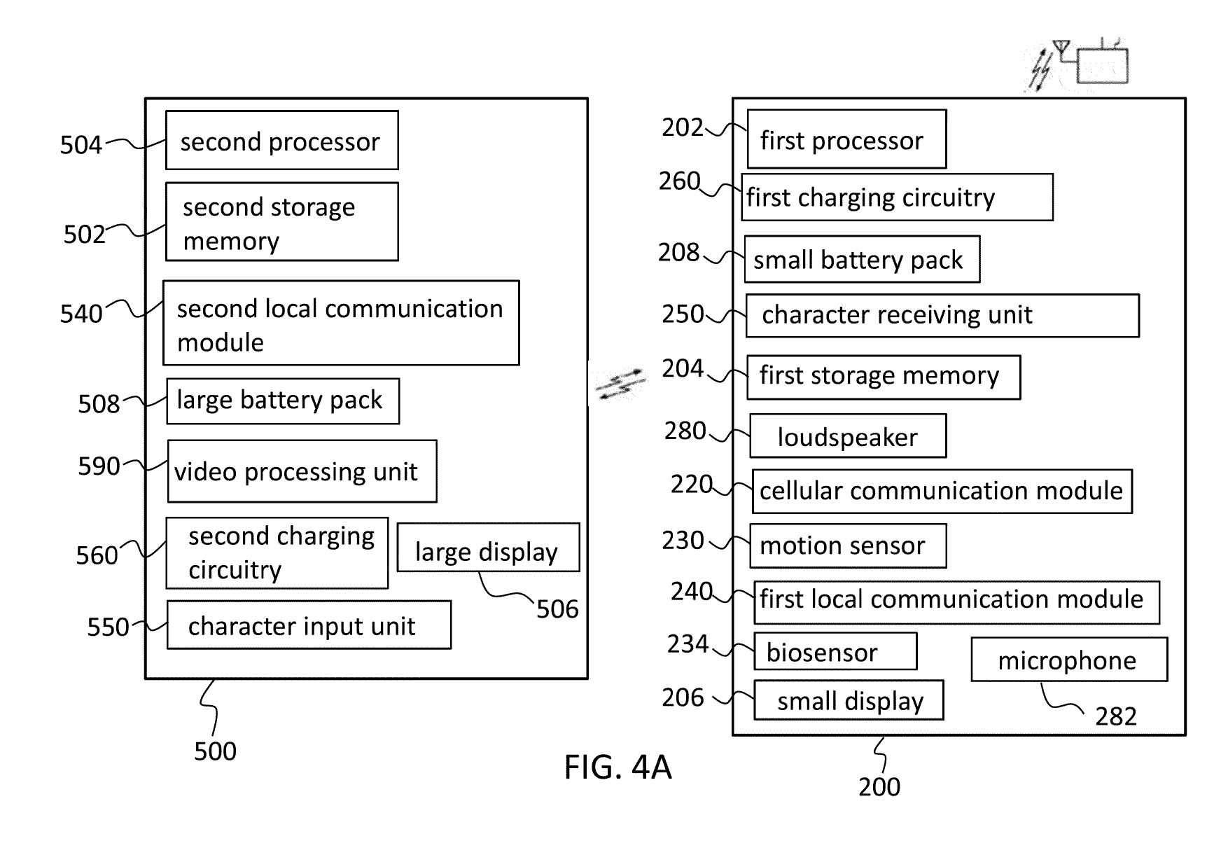

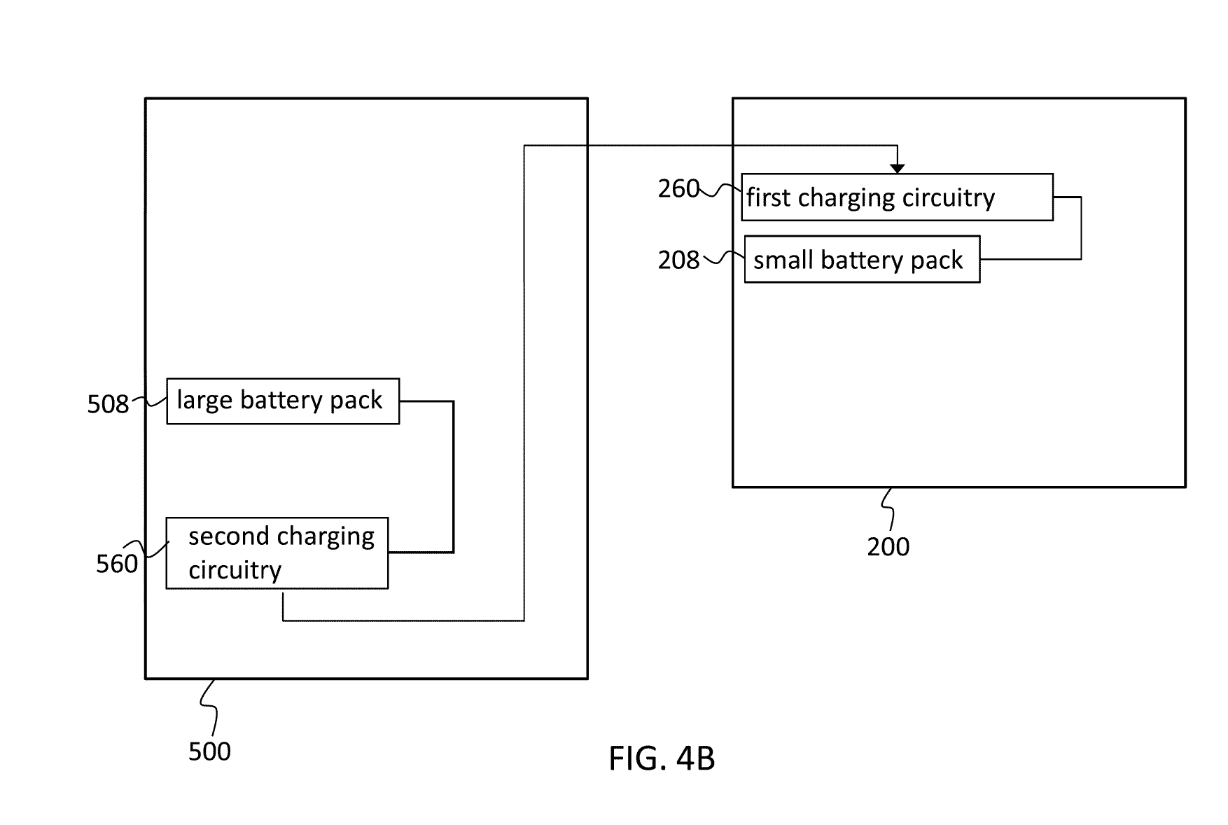

A first embodiment of the present invention is a cellular telephone set including a wearable electronic device and a display device. As shown in FIGS. 3, 4A, and 4B, the cellular telephone set of the first embodiment includes a wearable electronic device 200 and a display device 500. The display device 500 is a customized display device, meaning that it could be a specifically designed and manufactured display device to work with the wearable electronic device 200, or it could also be a general display device but installed with specific software. The wearable electronic device 200 is portable, and is physically configured to be suitable for wearing on the body of a user, such as being worn on the user's wrist in a way similar to a watch. The wearable electronic device 200 has the capability to perform basic cellular phone functions without using the display device 500 (i.e. when operating in a stand-alone mode), and the basic cellular phone functions include taking incoming calls, retrieving phone book data, making outgoing calls, receiving and displaying text messages, etc. Referring to the block diagram of FIG. 4A, the wearable electronic device 200 includes a first processor 202, a first storage memory 204 (which can be, for example, a flash memory), a small display 206, a small battery pack 208, a cellular communication module 220, and a first local communication module 240.

The small display 206 can be configured to display various information (e.g. time, text messages, icons indicating status of the wearable electronic device, and/or icons indicating the status of the customized display device), or to display icons for user to enable or disable specific functions. In this embodiment, the small display 206 cannot display a full QWERTY keyboard, and has only limited multimedia functionality (e.g. may provide audio reproduction, but provided no or only minimal video playback reproduction).

The small battery pack 208 is small in size, so as to be physically incorporated into the wearable electronic device 200. The small battery pack 208 is configured to receive power charging by the display device 500 in a wired or wireless manner. The capacity of the small battery pack 208 can be, for example, 500 to 800 mAh.

Again referring to FIGS. 3 and 4A, the cellular communication module 220 is to provide cellular communication between the wearable electronic device 200 and the nearby base station(s) provided by operators (e.g. Verizon, AT&T, China Mobile . . . ) via GSM/EDGE/CDMA/WCDMA/HSPA/LTE . . . standards. The cellular communication module 220 may include an antenna, an RF transceiver, and a baseband processor (which are not shown in the drawings).

The first local communication module 240 is included in the wearable electronic device 200 to provide local connection with the peripherals (e.g. Wifi hot spots or Bluetooth headset) and point-to-point wireless communication (e.g. Wifi Direct, Bluetooth) with the display device 500. Connection with the peripherals (e.g. Wifi hot spots or Bluetooth headset) and the display device 500 may utilize the same communication standards (for example, both use Bluetooth standard), or may also utilize different communication standards (for example, Bluetooth standard for the peripherals and Wifi Direct standard for the display device 500. The first local communication module 240 may include an antenna, an RF transceiver, and a baseband processor.

In the illustrated embodiment of FIG. 4A, the wearable electronic device 200 further includes a character receiving unit 250, a loudspeaker 280, a microphone 282, a first charging circuitry 260, and a motion sensor 230.

The character receiving unit 250 is configured for receiving data and/or character and/or alphanumeric inputs (for brevity, referred to as “data entry”) from the display device 500 in real time. The loudspeaker 280 is configured to produce sound for phone calls, or generate audio alarm or notifications for various applications operating on the wearable electronic device 200 or the display device 500. The microphone 282 is configured to receive sound for phone calls, or receive voice commands from the user to retrieve phone book stored in the wearable electronic device 200 and make outgoing calls. As shown in FIG. 4B, the first charging circuitry 260 is coupled to the small battery pack 208, so as to receive power wiredly or wirelessly from a second charging circuitry 560 coupled to a large battery pack 508 of the display device 500, and to charge the small battery pack 208.

The wired charging of the small battery pack 208 may be achieved through, for example, a micro-USB charging interface (not shown) incorporated within the first charging circuitry 260. Meanwhile, the wireless charging of the small battery pack 208 may be achieved through, for example, inductive charging circuitry incorporated within the first charging circuitry 260.

In this embodiment, the wearable electronic device 200 also includes a biosensor 234. The biosensor 234 is capable of detecting the user's body and generating physiological signals indicating the user's physical conditions. The physiological signals from the biosensor 234 may be further accumulated and analyzed on the wearable electronic device 200 or on the display device 500 to obtain the user's health information, such as the heart rate, or blood pressure . . . etc. The user's health information may be transmitted from the wearable device 200 to the display device 500, or vice versa, so that the user's health information may be displayed on wearable electronic device 200 in a first format and displayed on the display device 500 in a second format which is more content-rich and contains more information. In one embodiment, the biosensor 234 utilizes NPNS (Nanosecond Pulse Near-field Sensing) technology to detect the physical movements of a human body organ and generate signals which may be accumulated and analyzed to estimate the user's heart rate and blood pressure. The biosensor 234 in this embodiment, may be implemented in the form of the monitoring apparatus as disclosed in European patent publication no. EP1803396 B1 (of Application no. EP20060000082) which is titled “Monitoring Apparatus for Physical Movements of a Body Organ and Method for the Same” (contents of which is incorporated by reference herein in its entirety). The biosensor 234 may also be implemented as the measuring device illustrated in the IEEE paper “Using dual-antenna nanosecond pulse near-field sensing technology for non-contact and continuous blood pressure measurement,” authored by Hong-Dun Lin, Yen-Shien Lee, and Bor-Nian Chuang, and published in “Engineering in Medicine and Biology Society (EMBC), 2012 Annual International Conference” of the IEEE (Print ISBN: 978-1-4244-4119-8). The biosensor 234 includes a plurality of antennas (not shown) and a corresponding circuitry to radiate ultra-wideband electromagnetic waves, receive the ultra-wideband electromagnetic waves scattered by the body organ, acquire analog signals representing the physical movement of the body organ, and digitize the analog signals to generate the physiological signals indicating the user's physical conditions. It is noted that since such biosensor 234 makes use of Doppler effects to calculate and generate the physiological signals, therefore the detection result may be more reliable and accurate when the biosensor 234 is stationary so that there is less interference signals to be occurred simultaneously.

The wearable electronic device 200 further includes a motion sensor 230. The motion sensor 230 is capable of detecting movement of the wearable electronic device 200 and generating signals to the first processor 202 for determining whether the wearable electronic device 200 is in an environment suitable for the biosensor 234 to detect the body and generate the physiological signals. For example, when the wearable electronic device 200 is stationary, it may be more appropriate for the biosensor 234 to perform detection and generate physiological signals. The motion sensor 230 may include an accelerometer, a gyroscope or a magnetometer, or a combination of any two or three of the accelerometer, gyroscope, and magnetometer.

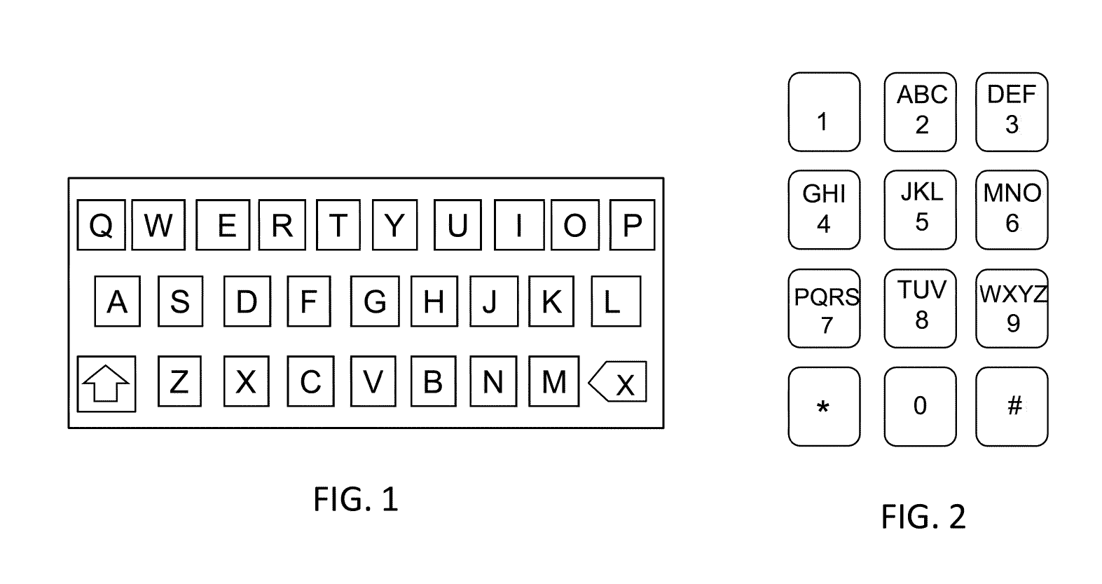

Referring again to the block diagram shown in FIG. 4A, the display device 500 of the cellular telephone set of the first embodiment includes a second processor 504, a second storage memory 502 (which can be, for example, a flash memory, configured for storing data), a large display 506, a large battery pack 508, a second local communication module 540, a character input unit 550, a video processing unit 590, and a second charging circuitry 560. The capacity of the large battery pack 508 can be, for example, 3000 to 6000 mAh, so the large battery pack 508 can provide sufficient power capacity for the large display 506 as well as for charging the wearable electronic device 200. The second local communication module 540 (which supports, for example, Wifi Direct or Bluetooth standards) is configured to provide point-to-point wireless communication with the wearable electronic device 200. The second local communication module 540 may include an antenna, an RF transceiver, and a baseband processor (not shown). The character input unit 550 provides a full QWERTY keyboard that can be implemented as a physical keyboard for data entry in real time. The full QWERTY keyboard may also be implemented as a virtual keyboard provided on the large display 506 with touch panel capability for data entry in real time. (See FIG. 1) The large display 506 has touch panel capability, so the display device 500 may further provide touch and/or multi-touch gesture operations for the user to control the display device itself (for example, control the display device to zoom-in or zoom-out a photo when the display device is showing a series of photos), or control the wearable electronic device through the point-to-point wireless communication (for example, control the wearable electronic device to pause or fast forward a piece of audio information when the wearable electronic device is reproducing it). The video processing unit 590 is configured to decode video data (e.g. MPEG-encoded bit stream or file) and reproduce the decoded video data on the large display 506. The second charging circuitry 560, coupled to the large battery pack 508, is configured to retrieve power from the large battery pack 508, and to charge the small battery pack 208 of the wearable electronic device 200 in a wired or wireless manner.

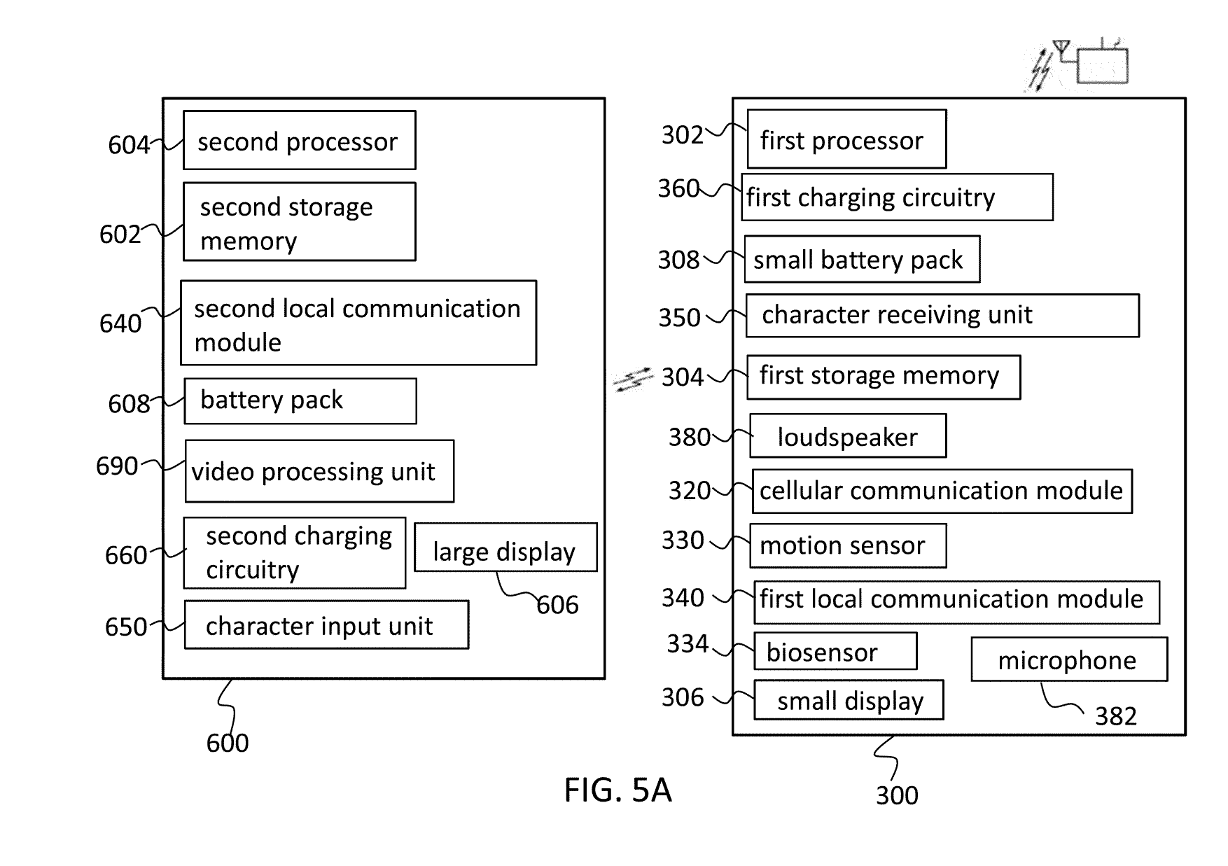

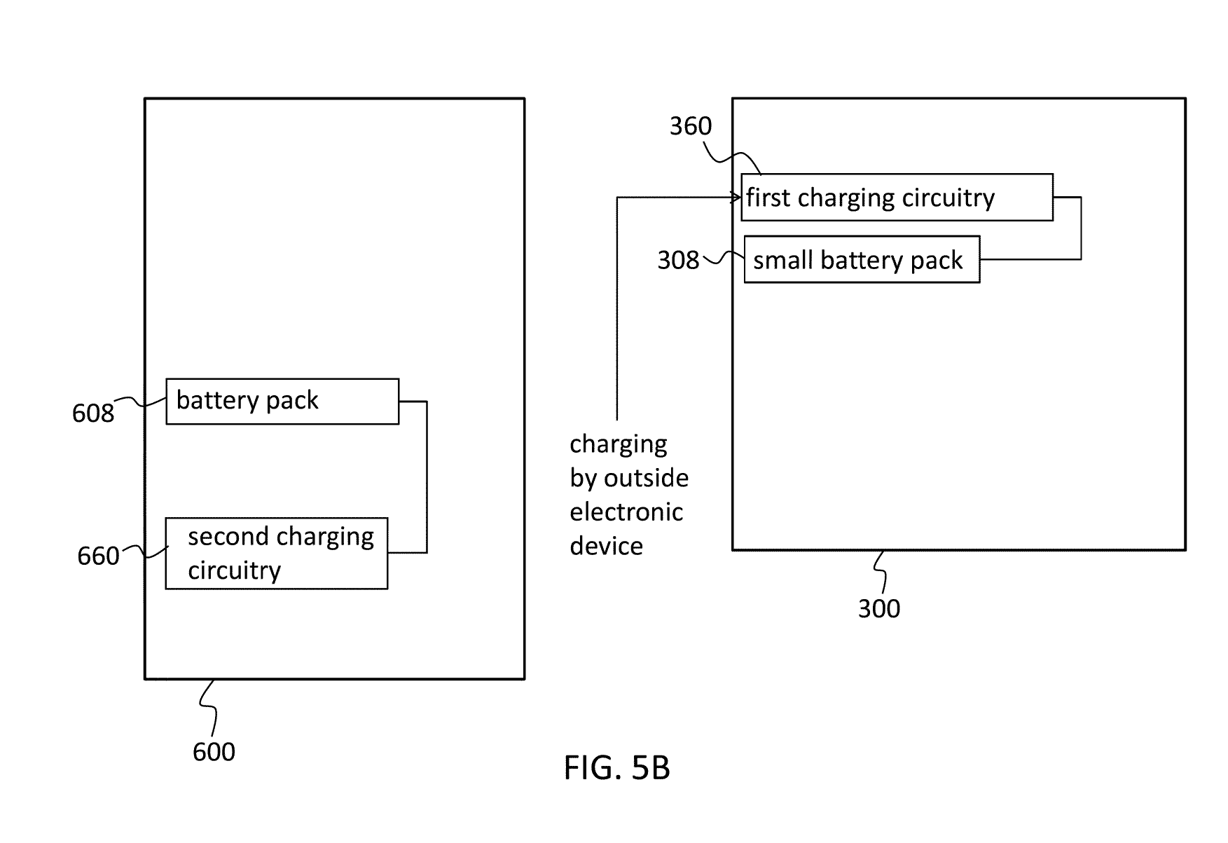

FIG. 5A and FIG. 5B illustrates a second embodiment of the cellular telephone set. The cellular telephone set includes a wearable electronic device 300 and a display device 600. The display device 600 is a customized display device, meaning that it could be a specifically designed and manufactured display device to work with the wearable electronic device 300, or it could also be a general display device but installed with specific software. The wearable electronic device 300 is portable and physically configured for wearing on the body of a user, such as being worn on the user's wrist in a way similar to a watch. The wearable electronic device 300 has the capability to perform basic cellular phone functions without using the display device 600 (i.e. when operating in a stand-alone mode), and the basic cellular phone functions include taking incoming calls, retrieving phone book data, making outgoing calls, receiving and displaying text messages, etc. Referring to FIG. 5A, the wearable electronic device 300 includes a first processor 302, a first storage memory 304 (which can be, for example, a flash memory, configured for storing data), a small display 306, a small battery pack 308, a cellular communication module 320, and a first local communication module 340. The small display 306 can be configured to display various information (e.g. time, text messages, icons indicating status of the wearable electronic device, and/or icons indicating the status of the display device), or to display icons for user to enable or disable specific functions. The wearable electronic device 300 provides a simple keyboard other than a full QWERTY keyboard. The simple keyboard may be a 12-key keypad, reduced-key keyboard (see FIG. 2), RIM's SureType keyboard, Jiugongge, and other similar reduced size keyboards. The simple keyboard may be implemented as a physical keyboard, or a virtual keyboard provided on the small display with touch panel capability. Referring to FIG. 5B, the small battery pack 308 is configured to receive power charging by an outside electronic device (e.g. a laptop or a portable power bank) in a wired manner via a micro USB connection, or in a wireless manner via inductive charging. The capacity of the small battery pack 308 can be, for example, 500 to 800 mAh.

The cellular communication module 320 is to provide cellular communication between the wearable electronic device 300 and the nearby base station(s) provided by operators (e.g. Verizon, AT&T, China Mobile, . . . etc.) via GSM/EDGE/CDMA/WCDMA/HSPAILTE . . . standards. The cellular communication module 320 may include an antenna, an RF transceiver, and a baseband processor (which are not shown in the drawings).

The first local communication module 340 is included in the wearable electronic device 300 to provide local connection with the peripherals (e.g. Wifi hot spots or Bluetooth headsets . . . ) and point-to-point wireless communication (e.g. Wifi Direct, Bluetooth) with the customized display device 600. The first local communication module 340 may include an antenna, an RF transceiver, and a baseband processor.

In the illustrated embodiment of FIG. 5, the wearable electronic device 300 further includes a character receiving unit 350, a loudspeaker 380, a microphone 382, a first charging circuitry 360, and a motion sensor 330.

The character receiving unit 350 is configured for receiving data and character entry input signals from the customized display device 600 in real time. The loudspeaker 380 is configured to produce sound for phone calls or generate audio alarm or notifications for various applications. The microphone 382 is configured to receive sound for phone calls, or receive voice commands from the user to take incoming calls or retrieve phone book to make outgoing calls. The first charging circuitry 360 is configured to be coupled to the small battery pack 308, to receive power from an outside power source through a conventional battery charger adapter so as to provide charging to the small battery pack 308 in a wired or wireless manner accordingly.

The wired charging of the small battery pack 308 may be achieved through, for example, a micro-USB charging interface. Meanwhile, the wireless charging of the small battery pack 308 may be achieved through, for example, inductive charging.

The motion sensor 330 of the wearable electronic device 300 is capable of detecting the movement of the wearable electronic device 300 and generating corresponding signals. The motion sensor 330 may include an accelerometer, or a gyroscope, or a magnetometer, or a combination of any two or three of the accelerometer, gyroscope, and magnetometer.

In the illustrated embodiment, the wearable electronic device 300 also includes a biosensor 334. The biosensor 334 is capable of detecting the user's body and generating physiological signals indicating the user's physical conditions. The physiological signals from the biosensor 334 may be further accumulated and analyzed on the wearable electronic device 300 or on the display device 600 to obtain the user's health information, such as heart rate, blood pressure . . . etc. The accumulation and analysis of the physiological signals may be performed on the wearable electronic device 300 or the display device 600 to obtain the user's health information. The user's health information may be transmitted between the wearable device 300 and the display device 600, so that the user's health information may be displayed on wearable electronic device 300 in a first format and displayed on the display device 600 in a second format which is more content-rich and contains more information. In one embodiment, the biosensor 334 utilizes NPNS (Nanosecond Pulse Near-field Sensing) technology to detect the physical movements of a human body organ and generate signals which may be accumulated and analyzed to obtain the user's heart rate and blood pressure. The biosensor 334 of this embodiment may be similar to the biosensor 234 of previous embodiment. The biosensor 334 includes a plurality of antennas (not shown) and a corresponding circuitry to radiate ultra-wideband electromagnetic waves, receive the ultra-wideband electromagnetic waves scattered by the body organ, acquire analog signals representing the physical movement of the body organ, and digitize the analog signals to generate the physiological signals. The wearable electronic device 300 may further includes a motion sensor 330 to detect movement of the wearable electronic device 300, so the first processor 302 may determine whether the wearable electronic device 300 is in a suitable environment for the biosensor 330 to detect the user's body and generate the physiological signals.

Referring again to the block diagram shown in FIG. 5A, the display device 600 of the cellular telephone set includes a second processor 604, a second storage memory 602 (which may be, for example, a flash memory) configured for storing data, a large display 606, a battery pack 608, a second local communication module 640, a character input unit 650, a second charging circuitry 660, and a video processing unit 690. The capacity of the battery pack 608 can be, for example, 3000 to 6000 mAh, so the battery pack 608 can provide sufficient power storage for the large display 606. The second local communication module 640 (which supports, for example, Wifi Direct or Bluetooth standard) is configured to provide point-to-point wireless communication with the wearable electronic device 300. The second local communication module 640 may include an antenna, an RF transceiver, and a baseband processor (not shown). The character input unit 650 provides a full QWERTY keyboard that can be implemented either as a physical keyboard or a virtual keyboard provided on the large display 606 with touch panel capability. The video processing unit 690 is configured to decode video data (e.g. MPEG-encoded bit stream or file) received from the wearable electronic device 300 via the local communication, and to reproduce the decoded video data on the large display 606. The second charging circuitry 660 is coupled to the battery pack 608, and configured to retrieve power from the battery pack 608. In some instances, the character input unit 650 having the full QWERTY keyboard can be used closely in tandem (side-by-side) along with the character receiving unit 350 for data entry in real-time for the wearable electronic device 300. In other words, data entry can be performed either through the full QWERTY keyboard at the display device 600 or through the reduced key keyboard (e.g., a 12-key keypad) at the small display 306 of the wearable electronic device 300, respectively. In another embodiment, the user may enable the video playback on the small display 306 to be directed to the large display 606 in real-time or near real-time. In such illustrated embodiment, the second local communication module 640 provides a local communication with the first local communication module 340 of the wearable electronic device 300, and vice versa; the encoded video data (e.g. MPEG encoded video data) is decoded on the wearable electronic device 300, and then transmitted to the display device 600 by the local communication for displaying on the large display 606. It is noted that after the encoded video data is decoded on the wearable electronic device 300, the decoded video data may be compressed on the wearable electronic device 300 before being transmitted to the display device 600 so as to fit into the bandwidth available for the local communication. In such case, the display device 600 would receive and decompress the decoded video data, and then display it on the large display 606. Since the video decoding is performed on the wearable electronic device 300, the display device 600 does not require high processing capability.

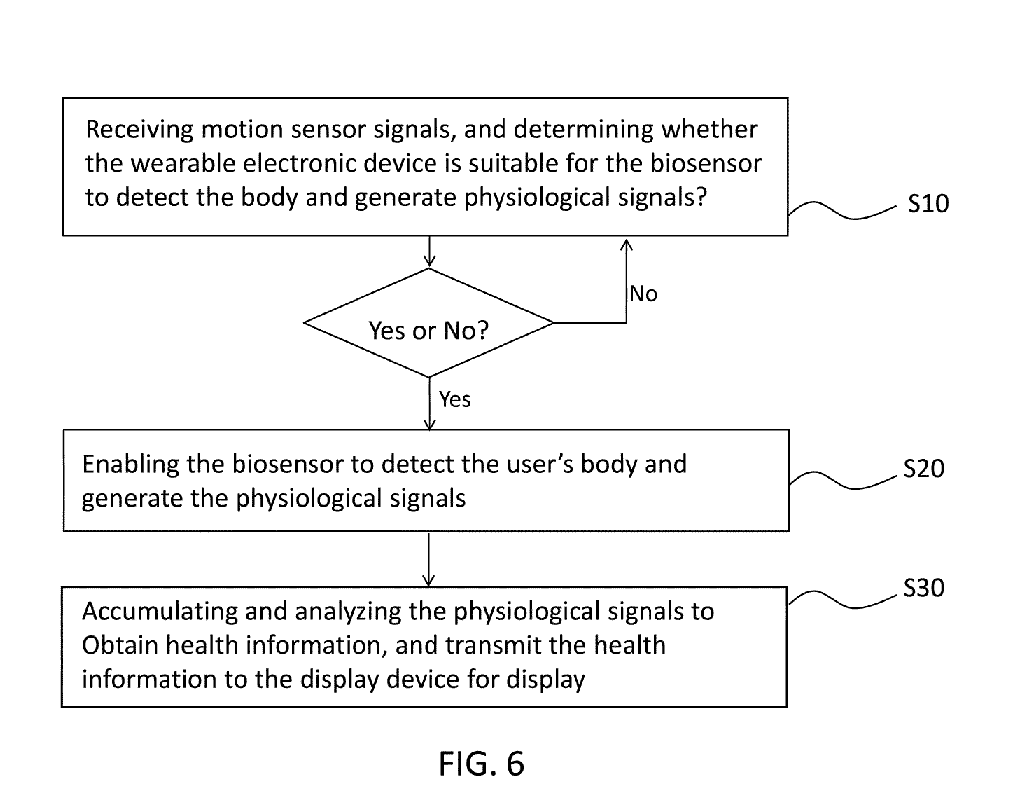

Referring to a flowchart of FIG. 6, an embodiment of a method to obtain the user's health information using the wearable electronic device 200 includes the following steps: Step S10: The first processor 202 of the wearable electronic device 200 receives signals from the motion sensor 230 and determines whether the wearable electronic device 200 is in an environment suitable for the biosensor to detect the body. If yes, go to Step S20; if no, return to Step S10. Step S20: The first processor 202 enables the biosensor 234 to detect the user's body for a predetermined period of time (which may be ranged, for example, from a few seconds to a few minutes), so as to collect enough data to generate physiological signals. Step S30: The physiological signals are accumulated and analyzed to generate the user's physical health information, and the health information is transmitted to the display device 500 for display. The health information may include the user's heart rate or blood pressure information, and may be stored and maintained in the second storage memory 502 of the display device 500.

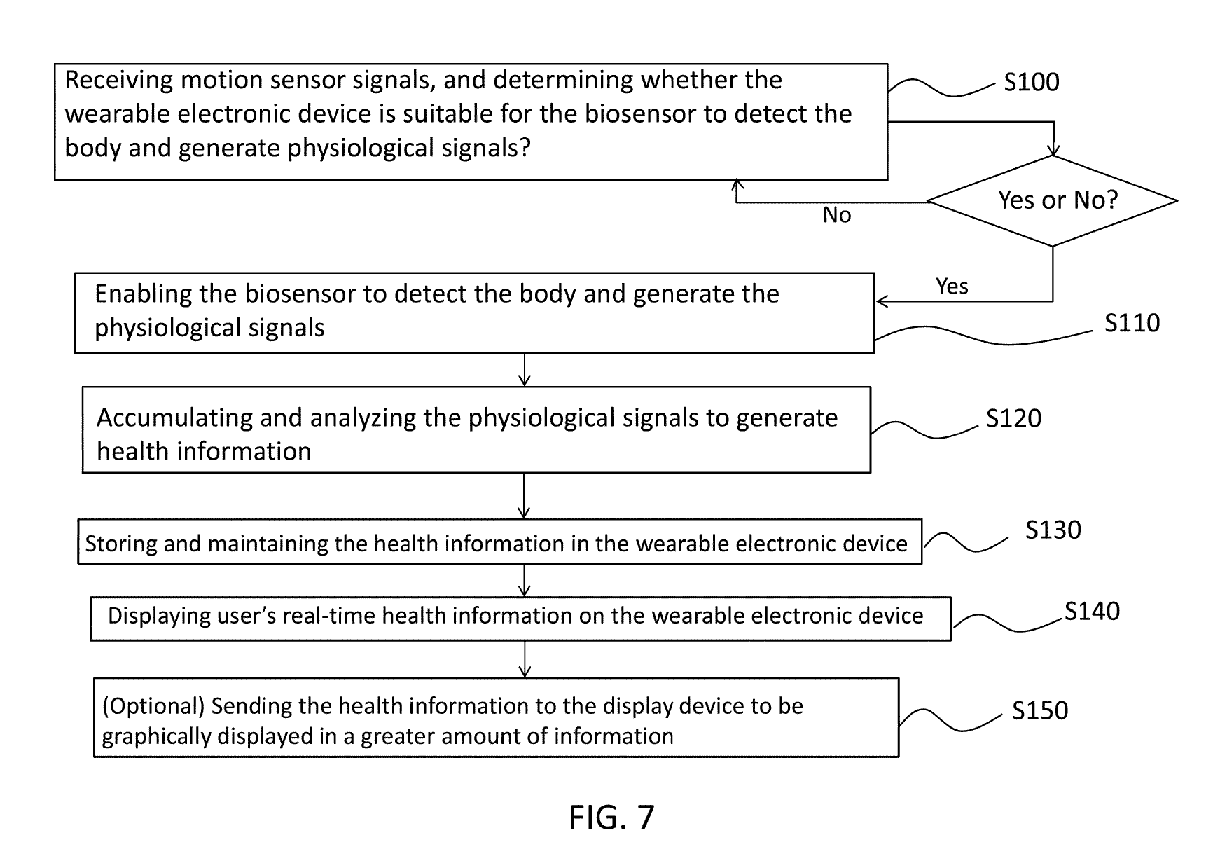

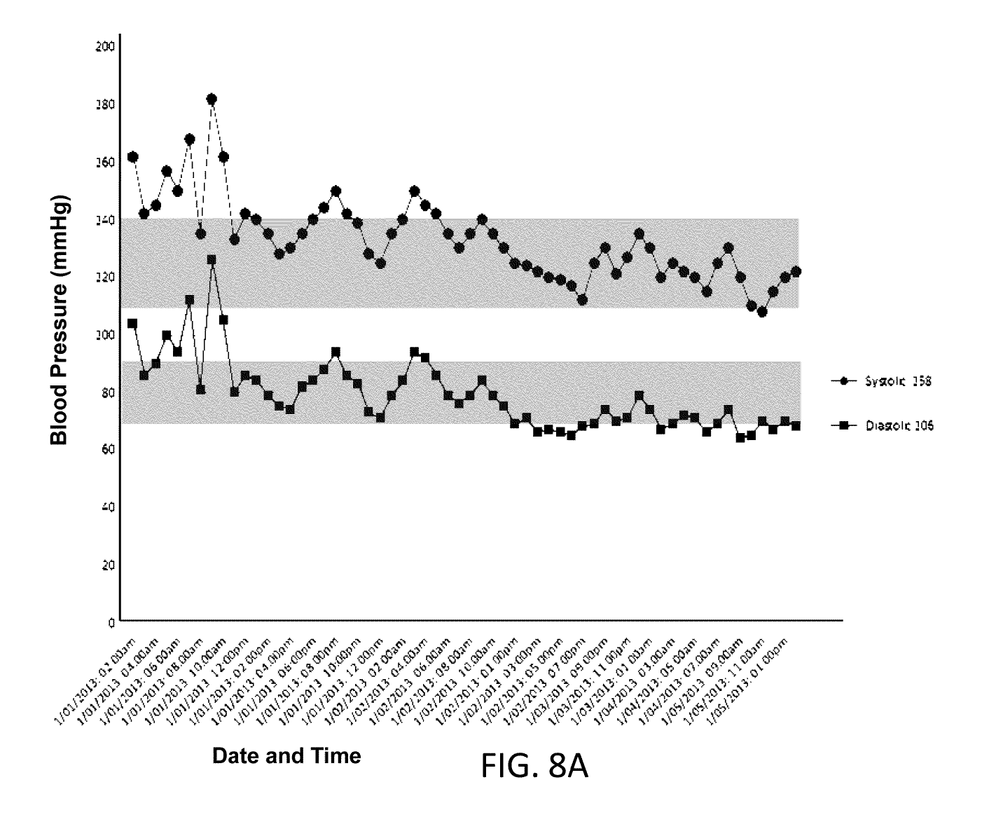

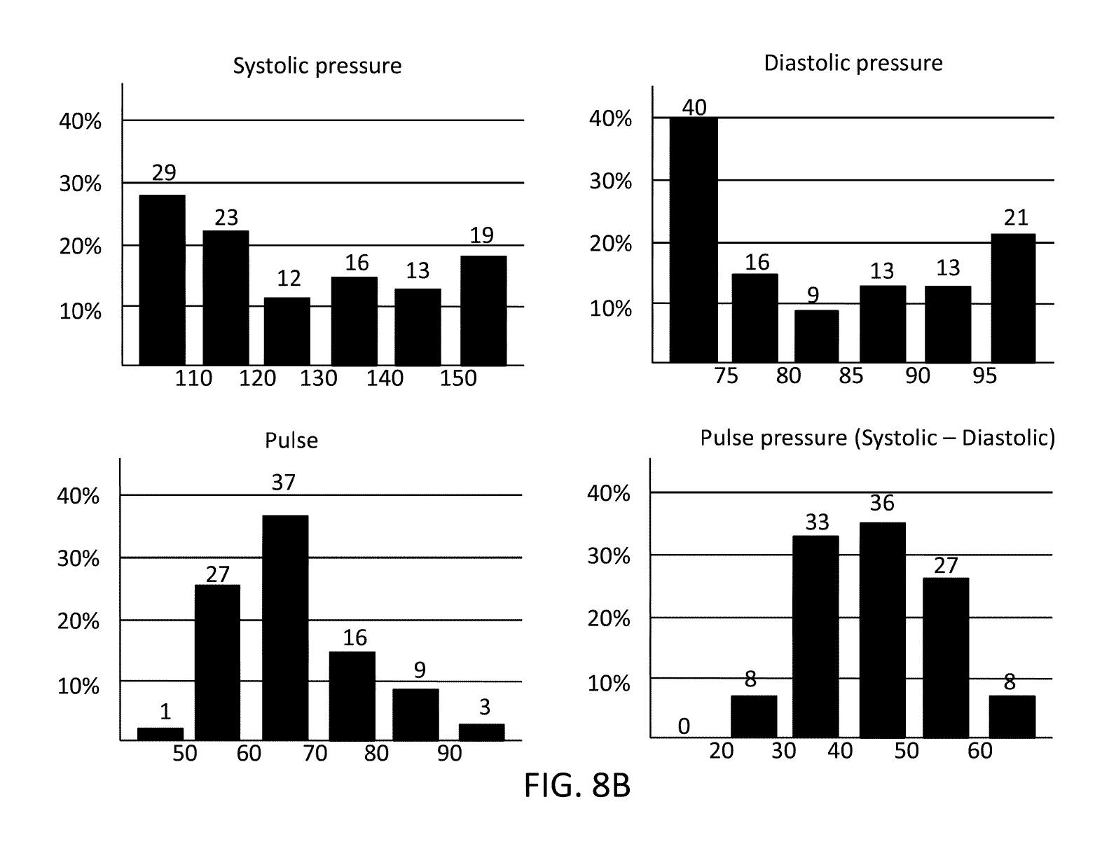

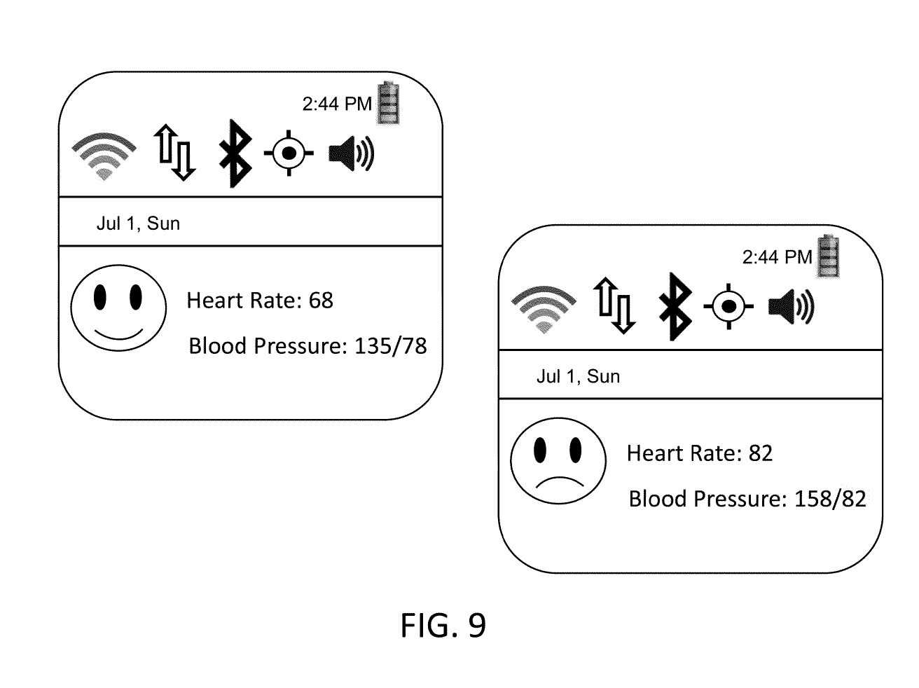

Another embodiment of a method to obtain the user's health information using the wearable electronic device 200 includes the following steps as shown in a flowchart in FIG. 7: Step S100: The first processor 202 of the wearable electronic device 200 receives signals from the motion sensor 230 and determines whether the wearable electronic device 200 is in a suitable environment for the biosensor 234 to detect the body and generate physiological signals. If yes, go to Step S110; if no, return to Step S100. Step S110: The first processor 202 enables the biosensor 234 to detect the user's body for a predetermined period of time (which may be ranged, for example, from a few seconds to a few minutes), so as to collect enough data to generate physiological signals from the user. Step S120: Accumulate and analyze the physiological signals to generate the user's health information, which may include the user's heart rate or blood pressure information, in the DRAM (Volatile memory, not shown) in the wearable electronic device 200. Step S130: Store and maintain the health information in the wearable electronic device 200. Step S140: The user's health information is displayed on the small display 206 of the wearable electronic device 200. Step S150: The first processor 202 may (optionally) also send the health information to the display device 500 so the health information may also be graphically displayed in a greater amount of information on the large display 506 as shown in FIGS. 8A and 8B as compared to that being displayed on the small display 206 of the wearable electronic device 200 as shown in FIG. 9. In this embodiment, only the latest detected health information (e.g. the currently detected heart rate and blood pressure information) is displayed on the small display 206 of the wearable electronic device 200. Additional health information, for example, the history record of the user's health information illustrated in various ways, is shown on the large display 506 of the display device 500. For example, FIG. 8A shows a detailed blood pressure versus date and time chart, and FIG. 8B shows various blood pressure and pulse histogram bar charts of the user based on data collected over a period of specified duration.

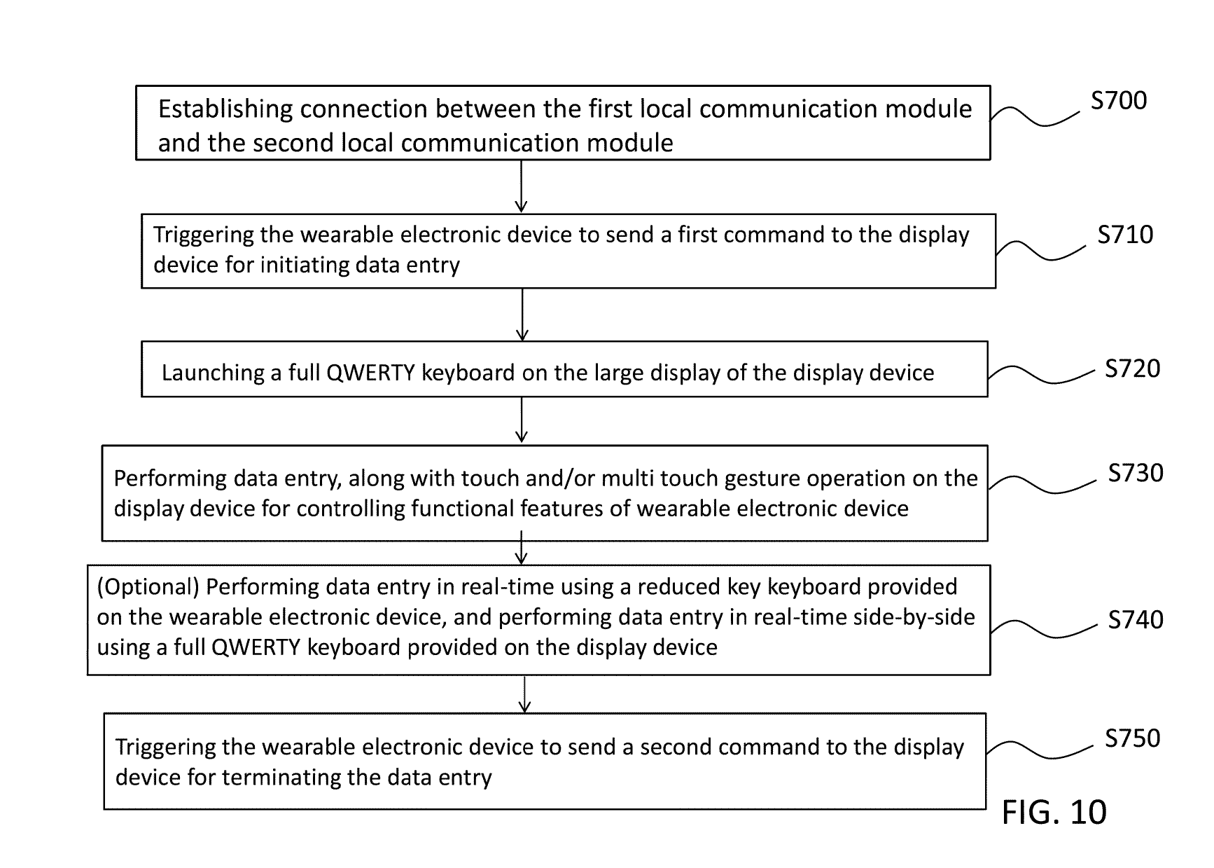

Referring to FIG. 5A and FIG. 10, an embodiment for a method for performing data entry for controlling various operating functions of the wearable electronic device include the following steps: Step S700: Establishing connection between the first local communication module and the second local communication module. Step S710: Triggering the wearable electronic device 300 to send a first command to the display device 600 for initiating data entry using the character input unit 650 of the display device 600. The triggering event may be, for example, the user to press a control button located on the wearable electronic device 300. The control button may be implemented as a physical button, or a virtual button displayed on the small display 306 with touch panel capacity. The triggering event may be, for example, the user to open an application on the wearable electronic device 300 which requires data entry. Step S720: Launching a full-scale virtual QWERTY keyboard on the large display 606 with touch panel capability. Step S730: Performing data entry, along with touch and/or multi touch gestures on the large display 606 by a user for controlling various functional features of the wearable electronic device 300, including but not limiting to, responding to real-time notifications, interacting to health information updates of the user, typing email messages and text messages, and transmitting the data entry to the wearable electronic device 300 in real time. Step S740: (Optional Step) Performing data entry in real-time using the character receiving unit 350 for the wearable electronic device 300 through the reduced key keyboard (i.e. 12-key keypad) at the small display 306 thereof while also performing data entry in real-time side-by-side using the virtual full QWERTY keyboard of the display device 600. Step S750: An event to trigger the wearable electronic device 300 to send a second command to the display device 600 for terminating the data entry on the virtual full QWERTY keyboard using the character input unit 650 of the display device 600. The terminating event may be, for example, the user to press the control button located on the wearable electronic device 300. The terminating event may be, for example, the user to close the software application which requires data entry.

Even though the display devices 500, 600 under several embodiments of the present invention have already been particularly described herein above, nevertheless the display device can be substituted by a conventional electronic device such as a computer, a laptop, a tablet PC, an iPad®, a smartphone, or the like, in other alternative embodiments. In addition, any of such conventional electronic devices can be also be modified or reconfigured to meet the structural, computing, and functional requirements of the illustrated customized display devices of the embodiments of present invention. Alternatively, the display device can be configured to be an simple and low-cost electronic device having a second processor that is based on a bare-bone mobile device ARM architecture processor, a second storage memory that can include just a small capacity of memory therein), a large display, a battery pack (that is smaller in power capacity than the large battery pack of the illustrated embodiments), a second local communication module, a character input unit, a video processing unit, and a second charging circuitry.

What is claimed is:

1. A cellular telephone set for communicating with a base station, comprising:
a wearable electronic device, comprising:
a first display;
a cellular communication module, for providing cellular communication with the base station;
a first local communication module;
a display device, which forms an independent body from the wearable electronic device, comprising:
a second display larger than the first display;
a second local communication module, for providing local communication with the first local communication module;
a character input unit, for providing a full QWERTY keyboard for data entry in real time by a user to the wearable electronic device,
wherein the wearable electronic device is physically configured for wearing by the user, and is capable for the user to take incoming calls and make outgoing calls without using the display device.

2. The cellular telephone set of claim 1, wherein the wearable electronic device further comprising a biosensor for detecting the user's body and generating physiological signals.

3. The cellular telephone set of claim 2, wherein the biosensor utilizes Nanosecond Pulse Near-field Sensing (NPNS) technology to detect physical movements of a body organ of the user, and to generate the physiological signals to be accumulated and analyzed to obtain the user's heart rate or blood pressure information.

4. The cellular telephone set of claim 1, wherein the wearable electronic device has no QWERTY keyboard capability, and the first display is configured to display time, text messages, icons indicating status of the wearable electronic device, or icons indicating status of the display device.

5. The cellular telephone set of claim 1, wherein the wearable electronic device includes a first battery pack and a first charging circuitry, the display device includes a second battery pack and a second charging circuitry, wherein the second battery pack has a larger capacity than the first battery pack, and the display device uses the second battery pack and the second charging circuitry to provide power charging to the wearable electronic device.

6. The cellular telephone set of claim 1, wherein the wearable electronic device further comprises a microphone to receive sound for a phone call or a voice command from the user, and a loudspeaker to produce sound for the phone call.

7. The cellular telephone set of claim 1, wherein the second display of the display device has touch panel capability, and the QWERTY keyboard is implemented as a virtual keyboard on the second display.

8. The cellular telephone set of claim 2, wherein the wearable electronic device further comprises a motion sensor capable of generating signals indicating movement of the wearable electronic device, and the biosensor is enabled to detect the user's body accordingly.

9. The cellular telephone set of claim 2, wherein the physiological signals are accumulated and analyzed on the display device to generate health information, the health information is transmitted to the wearable electronic device, the health information is shown on the wearable electronic device in a first format and is also shown on the display device in a second format having additional information than the first format.

10. The cellular telephone set of claim 2, wherein the physiological signals are accumulated and analyzed on the wearable electronic device to generate health information, the health information is shown on the wearable electronic device in a first format, and the health information is transmitted to the display device and is shown on the display device in a second format having additional information than the first format.

11. The cellular telephone set of claim 1, wherein the local communication between the wearable electronic device and the display device comprises Wifi Direct or Bluetooth standard.

12. The cellular telephone set of claim 1, wherein the wearable electronic device transmits video data to the display device via the local communication, and the display device displays the video data on the large display.

13. A wearable electronic device configured to be worn by a user, comprising:
a motion sensor for detecting movement of the wearable electronic device and generating motion signals;
a biosensor capable of detecting the user's body and generating physiological signals indicating the physical conditions, and
a processor configured to receive the motion signals and determine whether to enable the biosensor to detect the user's body and generate the physiological signals.

14. The wearable electronic device of claim 13, wherein the biosensor utilizes Nanosecond Pulse Near-field Sensing (NPNS) technology to detect physical movements of a body organ of the user, and to generate the physiological signals to be accumulated and analyzed to obtain the user's heart rate or blood pressure.

15. The wearable electronic device of claim 13, wherein the motion sensor includes an accelerometer, gyroscope, or magnetometer.

16. A method for a display device to provide data entry for a wearable electronic device, the wearable electronic device includes a first display, a cellular communication module for providing cellular communication, and a first local communication module, the display device forms an independent body from the wearable electronic device, the display device includes a second display and a second local communication module, the second display is larger than the first display and has touch panel capability, the method comprises:
establishing connection between the first local communication module and the second local communication module;
receiving a first command from the wearable electronic device to initiate the data entry;
launching a virtual full QWERTY keyboard on the second display of the display device;
receiving the data entry from a user and transmitting the data entry to the wearable electronic device in real time;
receiving a second command from the wearable electronic device to terminate the virtual full QWERTY keyboard on the second display of the display device.

17. The method of claim 16, further comprising:
receiving information from the wearable electronic device, and
displaying the information on the second display of the display device in a second format different from a first format of the information displayed on the wearable electronic device.

18. The method of claim 16, further comprising:
receiving physiological signals indicating the user's physical condition from the wearable electronic device;
accumulating and analyzing the physiological signals to obtain health information;
displaying the health information on the second display of the display device in a second format;
transmitting the health information to the wearable electronic device for display on the first display in a first format different from the second format.

19. The method of claim 18, wherein the physiological signals are generated by Nanosecond Pulse Near-field Sensing (NPNS) technology to detect physical movements of a body organ of the user.

20. The method of claim 16, wherein the connection between the first local communication module and the second communication module comprises Wifi standard.
